(19) United States
(12) Patent Application Publication
(10) Pub. No.: US 2003/0211154 A1
(43) Pub. Date: Nov. 13, 2003

(54) Coating composition for facilitating controlled release

(76) Inventors: Gour Mukherji, Gurgaon (IN); Manoj Kumar, Haryana (IN)

Correspondence Address: Jayadeep R Deshmukh, Ranbaxy Pharmaceuticals Inc, Suite 2100, 600 College Road East, Princeton, NJ, 08540, US

(21) Appl. No.: 10/276,366

(22) PCT Filed: May 7, 2001
(86) PCT No.: PCT/IB01/00777

(30) Foreign Application Priority Data
May 15, 2000 (IN) 514/DEL/2000

(51) Int. Cl. A61K 9/24; A61K 9/16; A61K 9/50

(52) U.S. Cl. 424/471

(57) ABSTRACT

The present invention relates to an extended release formulation comprising a coated drug containing core, wherein the coating is an aqueous coating, comprising an aqueous polymer dispersion of a water-insoluble film forming polymer in combination with an aqueous colloidal solution of a high viscosity swellable polymer.

### FIELD OF THE INVENTION

The present invention relates to an extended release formulation comprising a coated drug containing core, the coating being an aqueous coating, comprising an aqueous polymer dispersion of a water insoluble film forming polymer in combination with an aqueous colloidal solution of a high viscosity swellable polymer.

### BACKGROUND OF THE INVENTION

Most drugs are administered orally as tablets, capsules or other pharmaceutical dosage forms. However, in order to maintain therapeutic drug levels in the body, these drugs have to be administered three to four times a day. This dosage regimen is very inconvenient and leads to patient non-compliance.

Controlled release formulations which can deliver the drug over an extended period of time after administration not only provide a more patient friendly dosing regimen, but also maintain constant therapeutic levels of the drug in the blood thereby avoiding the crests and troughs associated with conventional immediate release dosage forms. Several techniques for delivering drugs at a constant rate, to control the site and duration of drug release, are known in the art.

One of the commonly used techniques for controlled drug delivery is the use of a sustained release coating. This technique is very versatile as it can be used for coating multiparticulate dosage forms, like, particles, granules and pellets, or unit dosage forms, like, tablets.

U.S. Pat. No. 4,894,240 describes a diltiazem pellet formulation where a core containing diltiazem and an organic acid is surrounded by multiple layers of a coating comprising a major proportion of a film forming water insoluble polymer and a minor proportion of a film forming water soluble polymer. The number of layers in the coating and the ratio of water soluble to water insoluble polymers controls the rate of release of diltiazem from the pellet core over a period of twenty four hours.

The process of coating described in this patent is very time consuming as several layers of coating needs to be applied to achieve an optimal coating and the pellets have to be dried after the application of each coat.

U.S. Pat. No. 5,840,332 describes a delivery system for targeted delivery comprising a core and coating. The coating is comprised of a water insoluble carrier with a water insoluble hydrophilic particle embedded in it to act as a channeling agent and thereby, produce an in-vitro dissolution rate faster than the coating comprising the water insoluble carrier only. The coating suspension is prepared preferably in ethanol. The coated product can be given for site-specific drug delivery and preferable area of treatment described in this patent is the ileum and the colon.

### SUMMARY OF THE INVENTION

It is one aspect of the present invention to describe an extended release formulation comprising a drug containing core coated with an aqueous coating composition wherein the coating composition comprises a polymer dispersion of a water insoluble film forming polymer in admixture with a colloidal solution of a high viscosity swellable polymer.

Another aspect of the present invention is to describe a process for the preparation of a pulsing release formulation comprising coating a drug containing core with an aqueous polymer dispersion of a water insoluble film forming polymer in admixture with a colloidal solution of a high viscosity swellable polymer.

The present invention makes it possible to extend the duration of drug release over an extended period of time, and also to release the drug in a site-specific manner. By changing the amounts of the two polymers, it is possible to achieve linear or pulsing release and the lag time of the pulsing release can also be controlled.

The coating composition of the present invention comprises a film-forming polymer, which is insoluble or minimally soluble in the gastric fluid, within which the high viscosity swellable polymer is dispersed. Upon contact with an aqueous media, the swellable polymer forms channels in the coating which allows the slow introduction of aqueous fluids into the core, thereby controlling the rate of initial drug release from the core. The site and duration of drug release can also be controlled by varying specific parameters such as the thickness of the outer coating and the amount of swellable material dispersed in the coating.

A variety of polymeric materials can be employed for film forming. Non-limiting examples of such film forming polymers include polymers selected from the group consisting of methacrylic acid copolymers—Types A, B and C, such as those sold under the trade name Eudragit L, Eudragit S and Eudragit L 100-55 from Rohm Pharma, methacrylate copolymers such as those sold under the trade name Eudragit N E, Eudragit R L, Eudragit R S and Eudragit F S from Rohm Pharma, cellulosic film forming polymers such as ethyl cellulose and sold under the trade names Surelease by Dow Chemicals and Aquacoat by FMC. Vinyl film forming polymers such as polyvinyl acetate and polyvinyl acetate phthalate may also be used as film forming polymers.

The swellable polymers used in combination with the film forming polymers are selected from the group consisting of polysaccharides, cross-linked polyacrylic acids and modified cellulose.

The polysaccharides that may be used in accordance with the present invention include those selected from the group consisting of xanthan gum, guar gum, locust bean gum and tragacanth gum. The swellable cross-linked polyacrylic acids include carbomers such as carbopol. Swellable cellulose such as hydroxypropyl cellulose, hydroxyethyl cellulose, hydroxypropyl methylcellulose and carboxymethylcelluloses may also be used in combination with the film forming polymers. The preferred swellable polymer is carbopol.

The swellable polymers, which may be used in combination with the film forming polymers, may be present in amounts upto 20% w/w of the film-forming polymer.

The coating composition may optionally contain other pharmaceutically acceptable excipients such as channeling agents, lubricants and plasticisers.

The channeling agent may be selected from the group consisting of lactose, starch and talc and may be present upto 50% or more preferably upto 30% of the film-forming polymer. The coating composition may also contain lubricants which function as anti-sticking agents. Lubricants may be selected from the group consisting of talc, colloidal silica and magnesium stearate. The lubricant quantity may be upto 200% or more, preferably upto 100% w/w, of the film forming polymer.

The coating composition may also contain suitable plasticizers which are selected from the group consisting of acetyl citrate, triacetin, acetylated monoglyceride, rape oil, olive oil, sesame oil, acetyltriethyl citrate, glycerin sorbitol, diethyloxalate, diethylmalate, diethylfumarate, dibutylsuccinate, dibutyl phthalate, dioctylphthalate, dibutylsebacate, triethylcitrate, tributylcitrate, glyceroltributyrate, glyceryl triacetate, polyethyleneglycol, propylene glycol, and mixtures thereof. The plasticizer is most preferably polyethylene glycol. The plasticizer may be present upto 40% w/w of the film forming polymer.

The composition of the present invention may be formulated into a dosage form suitable for oral administration, such as conventional whole tablet, chewable tablet, dispersible tablet, suspension or dry powder for reconstitution, sprinkles or other suitable oral dosage forms.

### DETAILED DESCRIPTION OF THE INVENTION

The following examples further illustrate the invention and are not intended to limit the scope of the invention.

### EXAMPLE 1

1.1 Drug Layering on Inert Core

| Ingredients | Amount (g) |
|---|---|
| Ciprofloxacin HCl | 597.5 |
| HP-L | 28.0 |
| Tal | 35.0 |
| DM water to | 1675 |

Procedure: Talc and hydroxypropyl cellulose was suspended in water followed by addition of ciprofloxacin hydrochloride and making up the volume with water. Resulting suspension was layered onto microcrystalline spheres (Celphere 102) using fluidized bed coating apparatus (Glaft-GPCG-1).

The drug-layered spheres were further coated with Eudragit L 30D-55 dispersions as discussed in section 1.2.

1.2 Coating on Drug Core

Amount (g)

| | Test- | |
|---|---|---|
| Ingredients | T50 | Control |
| Eudragit L30D-55 | 66.67 | 66.67 |
| Carbopol 971P (aqueous dispersion 1% w/w) | 40.0 | — |
| Polyethylene glycol | 3.06 | 3.0 |
| Talc (aqueous dispersion 40% w/w) | 25.5 | 25.5 |
| DM water to | 200.00 | 200.00 |

Aqueous colloidal solution of Carbopol and dispersion of Talc were mixed and the mixture was stirred into plasticized Eudragit dispersion with continuous stirring and made upto volume with remaining water. Resulting coating solutions containing 50% talc was sprayed in separate batches on the fluidized drug layered pellets (prepared according to experiment 1.1) to achieve a 12.5% w/w polymer application of the core. The drug layered pellets were also identically coated with “control” coating solution devoid of carbopol. When tested for dissolution, the “test” coated pellets showed a much greater effectiveness in controlling drug release in 0.1N hydrochloric acid media, as compared to the “control” which contained no carbopol in the coating solution, as shown in FIG. 1.

### EXAMPLE 2

| Ingredients | Amount (g) |
|---|---|
| Eudragit L30D-55 (30% dispersion) | 70.0 |
| Xanthan Gum (aqueous dispersion 1% w/w) | 42.0 |
| Polyethylene glycol | 3.21 |
| Talc (aqueous dispersion 30% w/w) | 71.4 |
| DM water to | 210.0 |

Colloidal solution of xanthan gum and talc dispersion was mixed and the resulting mixture was stirred into plasticized Eudragit dispersion, and suitably diluted with water. Resulting coating composition was sprayed over the drug layered pellets (prepared according to Example 1.1) using fluidized bed apparatus (Glatt GPCG-1) till 15% polymer application (by weight of the core) was achieved. When tested for dissolution, the coated pellets released 90% drug in 12 hours as shown in FIG. 2.

### EXAMPLE 3

3.1 Drug Layering on Inert Core

| Ingredients | Amount (g) |
|---|---|
| Cephalexin monohydrate | 109.00 |
| Talc IP | 7.00 |
| HPC-L | 10.9 |
| DM Water | 436.00 |

Talc and hydroxypropyl cellulose was suspended in sufficient water followed by addition of cephalexin monohydrate. The volume was made up to 436 g with water. Resulting suspension was coated over non-pareil beads by spraying in a fluidized bed equipment.

The drug-layered beads were coated with Eudragit RS30D dispersions as given in section 3.2.

3.2 Coating on Drug Core

Amount (g)

| Ingredients | Test | Control |
|---|---|---|
| Eudragit RS 30D | 66.7 | 66.7 |
| Triethyl citrate | 3.0 | 3.0 |
| Talc IP | 10.4 | 10.4 |
| Carbopol | 0.8 | — |
| DM water to | 200.00 | 200.00 |

Dilute hydrochloric acid solution was mixed with plasticized Eudragit dispersion. Aqueous colloidal solution of Carbopol and dispersion of Talc were mixed and the mixture was stirred into plasticized Eudragit dispersion with continuous stirring and made upto volume with remaining water. The “control” coating dispersion was prepared in an identical manner but without carbopol.

Resulting aqueous coating dispersion was sprayed over drug layered pellets (prepared as in Example 3.1) in a fluidized bed coater. A polymer application of 12.5% was achieved and the resulting pellets were subjected to dissolution testing in USP apparatus II, 50 rpm. FIG. 3 gives the extended release profiles in pH 6.8 phosphate buffer. The test product shows a greater control in drug release rate as compared to the control product indicating that this composition can control the rate of drug release even at very small percentage of polymer application.

### EXAMPLE 4

The coating composition can also be used on tablets as described in Example 4.

4.1 Tablet Formulation

| Ingredients | Amount (mg) |
|---|---|
| Celiprolol hydrochloride | 402.75 (400 mg drug) |
| Avicel 101 | 49.00 |
| Mannitol | 29.25 |
| Ac-di-sel | 10.00 |
| Magnesium Stearate | 9.0 |
| Total | 500.0 |

The drug and inactive excipients were mixed granulated and compressed to tablets and then coated with the coating composition described in Table 4.2.

4.2 Coating on the Tablet

| | Amount |
|---|---|
| Ingredients | (mg) |
| Ethyl cellulose (Aquacoat ECD - 30) | 92.15 |
| Tri-ethyl Citrate | 1.0 |
| Carbopol 934 P (as 1% w/w aq. solution) | 5.0 |
| Purified water | 86.5 |

Carbopol solution was stirred into a plasticized dispersion of ethyl cellulose and made upto volume with remaining water. The composition was coated on the tablets to a polymer application of 5% w/w. The resulting tablets were subject to dissolution testing in USP apparatus 11 at 50 rpm in pH 6.8 phosphate buffer. The dissolution profile given in FIG. 4 shows that the tablets exhibit a significant lag time in drug release indicating that this system can also be used for pulsing delivery of drugs.

While the present invention has been described in terms of its specific embodiments, certain modifications and equivalents will be apparent to those skilled in the art and are intended to be included within the scope of the present invention.

We claim:

1. An extended release formulation comprising: a drug containing core coated with an aqueous coating composition, wherein the coating composition comprises a polymer dispersion of a water insoluble film forming polymer in combination with a high viscosity swellable polymer.

2. The formulation of claim 1 wherein the water insoluble, film forming polymer is selected from the group consisting of methacrylic acid copolymers, methacrylate copolymers, cellulosic polymers and vinyl polymers.

3. The formulation of claim 2, wherein the cellulosic film-forming polymer is ethyl cellulose.

4. The formulation of claim 2, wherein the vinyl film-forming polymer is selected from polyvinyl acetate phthalate and polyvinyl acetate.

5. The formulation of claim 1 wherein the high viscosity swellable polymer is selected from the group consisting of polysaccharides, cross linked polyacrylic acid and modified cellulose.

6. The formulation of claim 7 wherein the polysaccharide is selected from the group consisting of xanthan gum, guar gum, locust bean gum and tragacanth gum.

7. The formulation of claim 7 wherein the cross-linked polyacrylic acid is carbomer.

8. The formulation of claim 7 wherein the modified cellulose are selected from the group consisting of hydroxypropylcelluse, hydroxyethylcellulose, hydroxypropyl methyl cellulose and carboxymethyl cellulose.

9. The formulation of claim 1 wherein the high viscosity swellable polymer is present upto 20% w/w of the film-forming polymer.

10. The formulation of claim 1, wherein one or more channeling agents are present.

11. The formulation of claim 12, wherein the channeling agents are selected from the group consisting of lactose, starch and talc.

12. The formulation of claim 12, wherein the channeling agent is present upto 50% or more preferably upto 30% w/w of polymers.

13. The formulation of claim 1, wherein one or more lubricants are present.

14. The formulation of claim 15, wherein the lubricants are selected from the group consisting of talc, colloidal silica and magnesium stearate.

15. The formulation of claim 15, wherein the lubricants are present upto 200% or more preferably upto 100% w/w of the film-forming polymer.

16. The formulation of claim 1, wherein plasticizers are incorporated in the composition.

17. The formulation of claim 18, wherein the plasticizers are selected from the group consisting of acetyl citrate, triacetin, acetylated monoglyceride, rape oil, olive oil, sesame oil, acetyltriethyl citrate, glycerin sorbitol, diethyloxalate, diethylmalate, diethylfumarate, dibutylsuccinate, dibutyl phthalate, diethylmaloate, dioctylphthalate, dibutylsebacate, triethylcitrate, tributylcitrate, glyceroltributyrate, glyceryl triacetate, polyethyleneglycol, propylene glycol, and mixtures thereof.

18. The formulation of claim 18, wherein the plasticiser is preferably polyethylene glycol (PEG).

19. The formulation of claim 18 wherein the plasticizer is present upto 40% w/w of the film-forming polymer.

20. The formulation of claim 1, wherein the dosage form is designed for pulsing delivery of the drug.

21. The formulation of claim 1, formulated as a dosage form suitable for oral administration.

22. The formulation of claim 23, wherein the formulation suitable for oral administration may be conventional whole chewable tablet, dispersible tablet, suspension or dry powder for reconstitution, for sprinkle or any other suitable oral dosage form.
